(19) United States
(12) Patent Application Publication
(10) Pub. No.: US 2004/0254598 A1
(43) Pub. Date: Dec. 16, 2004

(54) Suture cutter

(76) Inventors: Brian S. Schumacher, Orlando, FL (US); Mark A. Goldin, Orlando, FL (US)

Correspondence Address: FROST BROWN TODD, LLC, 2200 PNC CENTER, 201 E. FIFTH STREET, CINCINNATI, OH, 45202, US

(21) Appl. No.: 10/869,568

(22) Filed: Jun. 16, 2004

Related U.S. Application Data
(60) Provisional application No. 60/478,796, filed on Jun. 16, 2003.
(60) Provisional application No. 60/552,020, filed on Mar. 10, 2004.

(51) Int. Cl. A61B 17/04

(52) U.S. Cl. 606/170

(57) ABSTRACT

A suture cutter assembly has a cutter blade axially reciprocable relative to a tube having at least one opening. An offset is in the tube or the cutter blade for capturing of the severed portion of the suture as the blade is advanced within the tube. A combined suture, bead, and knot cutter assembly includes a recess formed in a shaft which is axially reciprocable within a tube, which receives a suture enlargement, thereby placing the suture or suture enlargement to be cut by advancing the shaft. A suture may be passed through a distal opening in a tube and an aperture formed in the sidewall of the tube, permitting the pushing of a knot in the suture by the distal edge of the tube.

This application claims priority benefit of the disclosure of U.S. Provisional Patent Application Ser. No. 60/478,796, entitled “Suture Cutter and Suture Bead and Knot Cutter,” filed Jun. 16, 2003. This application also claims priority benefit of the disclosure of U.S. Provisional Patent Application Ser. No. 60/552,020, entitled “Suture Cutter and Knot Pusher,” filed Mar. 10, 2004.

### BACKGROUND OF THE INVENTION

The present invention relates generally to suture cutters, and is particularly directed to devices for cutting stainless steel sutures arthroscopically. The invention will be specifically disclosed in connection with a device that cuts a suture while capturing the severed segment. The invention will also be specifically disclosed in connection with a device that cuts a suture having a bead attached thereto while retaining the bead and severed segment.

The present invention also relates to knot-pushers, and includes, but is not limited to, devices capable of both pushing suture knots and cutting sutures arthroscopically.

Cutting sutures at the appropriate location can be difficult, particularly during procedures where access is limited, such as arthroscopic surgery. Capturing the severed portion of the suture can be equally difficult. The difficulties are increased when the suture material is hard to cut, such as stainless steel sutures.

Similar difficulties may be encountered when cutting a suture having an enlarged end, such as to remove a knot or an attached structure, such as a bead, from a suture. For example, such difficulties are encountered in removing a stop member from a suture adjacent an anchor as shown in United States Patent Publication 2002/0077631, filed Jun. 20, 2002, entitled “Apparatus And Methods For Tendon Or Ligament Repair,” the disclosure of which is incorporated herein by reference.

Pushing suture knots may also be difficult, particularly in close quarters such as those encountered at many surgical sites. In addition, the separate acts of pushing a suture knot and cutting a suture end typically require the use of more than one tool. The need for separate tools for these separate acts has its own obvious shortcomings.

### BRIEF DESCRIPTION OF THE DRAWINGS

The accompanying drawings incorporated in and forming a part of the specification illustrate several aspects of the present invention, and together with the description serve to explain the principles of the invention. In the drawings:

FIG. 1 is a top view of a suture cutter assembly constructed according to teachings of the present invention.

FIG. 2 is a cross sectional side view of the device shown in FIG. 1 taken along line 2-2 of FIG. 1.

FIG. 3 is an enlarged, fragmentary side view of the distal end of the tube of the device shown in FIG. 1.

FIG. 4 is an enlarged, fragmentary top view of the distal end shown in FIG. 3.

FIG. 5 is an enlarged, fragmentary side view of the opposite side of the distal end shown in FIG. 3.

FIG. 6 is an enlarged, fragmentary cross sectional view of the distal end of the cutter blade of the device shown in FIG. 1.

FIG. 7 is similar to FIG. 3, with a suture disposed in the slot.

FIGS. 8-10 are enlarged, fragmentary, cross sectional views of the suture cutter assembly of FIG. 1, taken along line 8-8 of FIG. 7, showing the cutter blade cutting a suture disposed in the slot.

FIG. 11 is an enlarged, fragmentary, cross sectional view similar to the view of FIG. 8 of an alternate embodiment of the slot and distal end of the cutter blade.

FIG. 12 is an enlarged, fragmentary, cross sectional view similar to the view of FIG. 10 of another alternate embodiment of the slot and distal end of the cutter blade.

FIG. 13 is a top view of a combined suture bead and knot cutter/suture cutter assembly constructed according to teachings of the present invention.

FIG. 14 is a cross sectional side view of the device shown in FIG. 13 taken along line 12-12 of FIG. 11.

FIG. 15 is an enlarged, fragmentary, cross sectional view of the distal end of the device shown in FIGS. 13 and 14.

FIG. 16 is an enlarged, fragmentary side view of the distal end of the cutter tube showing the opening for the suture bead and knot cutter.

FIG. 17 is a top view of the distal end shown in FIG. 16.

FIG. 18 is an enlarged, fragmentary side view of the distal end of the cutter blade of the device shown in FIGS. 13 and 14.

FIG. 19 is an enlarged, fragmentary top view of the distal end shown in FIG. 18.

FIG. 20 is an enlarged, fragmentary side view of the distal end shown in FIG. 18, illustrating the suture bead and knot cutter blade.

FIG. 21 is an enlarged, fragmentary side view of the distal end shown in FIG. 18, illustrating the suture cutter blade.

FIG. 22 is an enlarged, fragmentary cross sectional side view showing a bead disposed in the suture bead and knot cutter portion of the device shown in FIGS. 13 and 14, prior to cutting the suture.

FIG. 23 is an enlarged, fragmentary side view showing the cutting edges adjacent each other, as the suture is being severed.

FIG. 24 is an enlarged, fragmentary cross sectional side view showing a bead disposed in the suture bead and knot cutter after cutting the suture.

FIG. 25 is an enlarged, fragmentary cross sectional side view similar to the view of FIG. 24 showing an alternate embodiment of the suture bead and knot cutter portion.

FIG. 26 is a top view of an alternate suture cutter assembly constructed according to teachings of the present invention.

FIG. 27 is an enlarged, fragmentary cross sectional side view of the device shown in FIG. 26 taken along the axis of the device, parallel to the page of FIG. 26, with the device in a pushing configuration.

FIG. 28 is an enlarged, fragmentary cross sectional side view of the device shown in FIG. 26 taken along the axis of the device, parallel to the page of FIG. 26, but with the device in cutting mode.

FIG. 29 is an enlarged cross sectional end view of the device shown in FIG. 26, but with the device in transition between a pushing configuration and cutting mode.

FIG. 30 is an enlarged, perspective, fragmentary view of the device shown in FIG. 26 in partial cross section in a transition between a pushing configuration and cutting mode.

FIG. 31 is an enlarged, fragmentary side view of the distal end of the tube of the device shown in FIG. 26.

FIG. 32 is an enlarged, fragmentary side view of the opposite side of the distal end shown in FIG. 31.

FIG. 33 is an enlarged, fragmentary cross sectional view of the distal end of the cutter blade of the device shown in FIG. 26.

FIG. 34 is an enlarged, fragmentary cross sectional view of the distal end of the device shown in FIG. 26 in a pushing configuration.

FIG. 35 is a perspective view of the device shown in FIG. 26 with a suture disposed within the device for pushing a knot.

FIG. 36 is similar to FIG. 31, with a suture disposed in the apertures for cutting.

FIGS. 37-39 are enlarged, fragmentary, cross sectional views of the suture cutter assembly of FIG. 26, taken along line 37-37 of FIG. 36, showing the cutter blade cutting a suture disposed in the apertures.

FIG. 40 is an enlarged, fragmentary, cross sectional view similar to the view of FIG. 37 of an alternate embodiment of the apertures and distal end of the cutter blade.

FIG. 41 is an enlarged, fragmentary, cross sectional view similar to the view of FIG. 39 of another alternate embodiment of the apertures and distal end of the cutter blade.

Reference will now be made in detail to the present preferred embodiment of the invention, an example of which is illustrated in the accompanying drawings.

### DETAILED DESCRIPTION OF AN EMBODIMENT OF THE PRESENT INVENTION

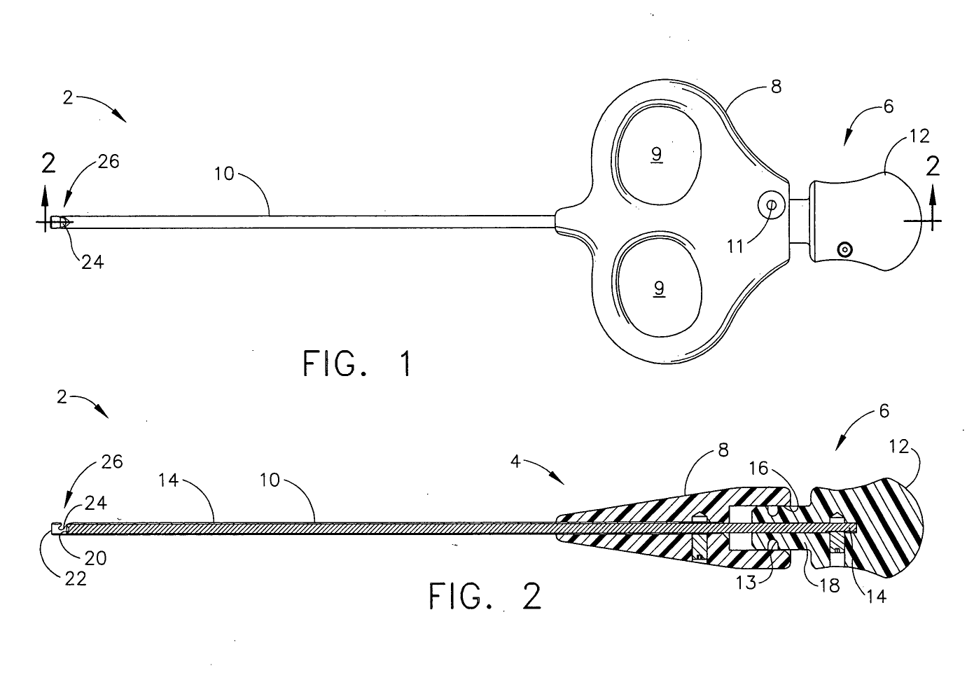

Referring now to the drawings in detail, wherein like numerals indicate the same elements throughout the views, FIG. 1 is a top view of suture cutter assembly 2 constructed according to the teachings of the present invention. FIG. 2 is a cross sectional view taken along line 2-2 of FIG. 1. Suture cutter assembly 2 includes tube assembly 4 and cutter assembly 6. Tube assembly 4 comprises grip 8 having two openings 9 for the operator's fingers. Grip 8 is secured to one end of hollow tube 10. Cutter assembly 6 includes knob 12 secured to one end of shaft 14. Although in the embodiment depicted, tube 10 is secured to grip 8 and shaft 14 is secured to knob 12 by respective set screws, any suitable means for securing them together may be used.

In the depicted embodiment, shaft 14 is rigid and solid, and dimensioned to reciprocate within rigid tube 10. The clearance between shaft 14 and tube 10 provides some resistance to axial movement of shaft 14. Grip 8 includes cylindrical recess 16 which is configured to receive extension 18 of knob 12.

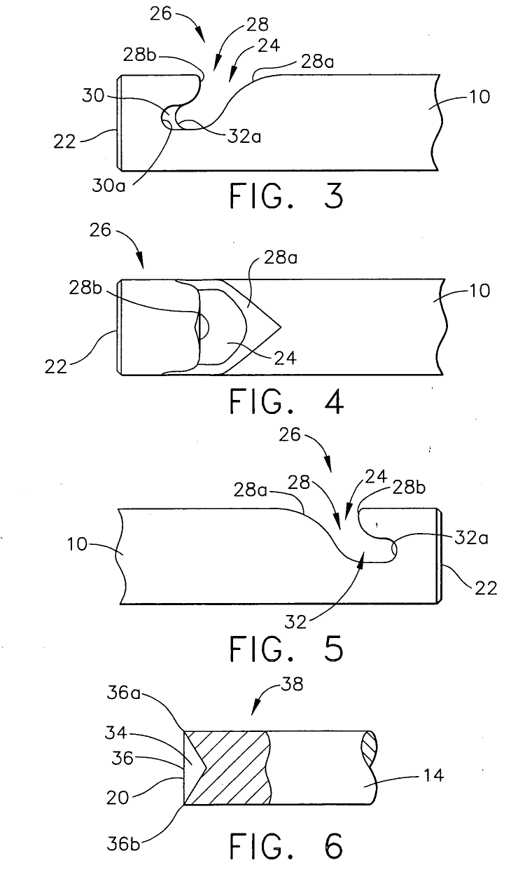

Referring also to FIGS. 3-5, slot 24 is formed through the sidewall of tube 10 at distal end portion 26 of tube 10 which communicates with the interior defined by tube 10. Slot 24 is shown as opening in the same direction as openings 9, providing an index to the relative orientation between grip 8 and opening 24. However, slot 24 may open in any direction relative to grip 8.

Slot 24, also referred to as a notch or opening, includes entrance portion 28 starting tangent to the outer surface of tube 10, which leads to aligned axial portions 30 and 32. This “J” notch configuration allows a suture to be hooked into and retained within the slot until it is cut. It is noted that the J notch configuration may be omitted, not having the axially extending portions, but still having edges disposed at transversely (to the longitudinal axis) spaced apart locations of the tube which present an opposing surface that limits downstream movement of the respective portions of the suture disposed adjacent thereto. The opposite edges 28a and 28b are not sharp. Axial portions 30 and 32 terminate in respective arcuate edges 30a and 32a, which are axially offset from each other as can be seen in FIG. 3, where edge 30a is disposed closer to end 22 than edge 32a. Slot 24 may be formed by any well known process, such as by milling or electro discharge machining.

As seen in FIG. 6, end 20 of shaft 14 comprises the cutter blade, which in the embodiment depicted comprises conical shape depression 34 forming an annular sharp edge 36 about the periphery of end 20 of distal end portion 38. Edge 36 comprises edge 36a and edge 36b.

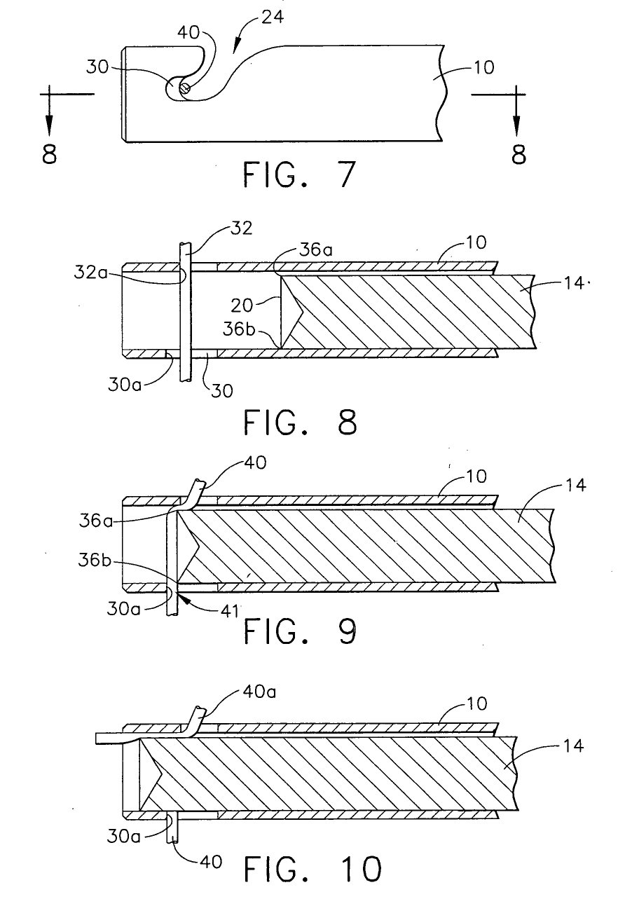

Referring to FIGS. 7-10, the process for cutting suture 40 with suture cutter assembly 2 is illustrated. Suture 40 is hooked by slot 24 so that suture 24 is disposed in axial portions 30 and 32 of slot 24, with end 20 and sharp edge 36 initially located up from of slot 24, away from suture 40, so that end 20 does not occlude any portion of slot 24. To assist in locating end 20 in the initial pre-cut position, grip 8 includes locating hole 11 extending completely therethrough, and knob 12 includes annular groove 13. The location of slot 24 relative to locating hole 11 and the location of end 20 relative to annular groove 13 are such that when annular groove 13 is aligned with locating hole 11, end 20 is in the initial pre-cut position, and the operator can see all the way through hole 11. Other configurations can be used to provide for the initial pre-cut positioning of end 20 such as by a detent.

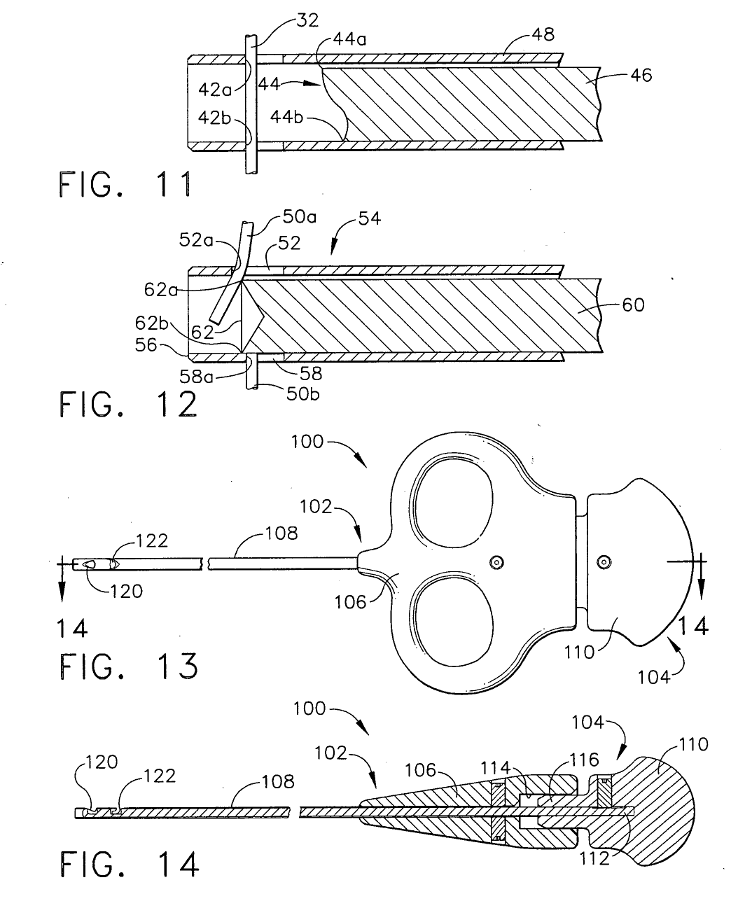

As seen in FIG. 8, suture 40 is shown disposed adjacent edge 32a, generally perpendicular to the longitudinal axes of tube 10 and shaft 14. However, it will be appreciated that suture 40, while disposed in slot 24 may be spaced from or adjacent to both edges 30a and 32a, until shaft 14 is urged into contacted with suture 40.

In FIG. 9, shaft 14 has been moved from the first position shown in FIG. 8, urged into contact with suture 40, with sharp edge 36a of end 20 disposed adjacent edge 32a, and occluding slot 24 adjacent edge 32a. The transverse clearance, also referred to as diametrical clearance, between shaft 14 adjacent edge 36a and the sidewall of tube 10 adjacent edge 32a, has a dimension which is less than a corresponding dimension of the suture, such that a portion of suture 40 is captured or retained between the interior surface the sidewall of tube 10 and shaft 14 but larger than a dimension what would sever suture 40. This configuration makes edge 32a the capture edge of slot 24. A bend is formed in suture 40 by this action, and shaft 14, and sharp edge 36b are concomitantly urged against the opposite side of the interior of tube 10, the portion adjacent cutting edge 30a.

At the position shown in FIG. 9, gap 41, extending longitudinally, is defined between edge 30a and edge 36b. At the position shown in FIG. 9, gap 41 has a longitudinal dimension which is smaller than the corresponding dimension of suture 40. Gap 41 may be smaller than the corresponding dimension when slot 24 adjacent edge 32a is initially occluded by end 20.

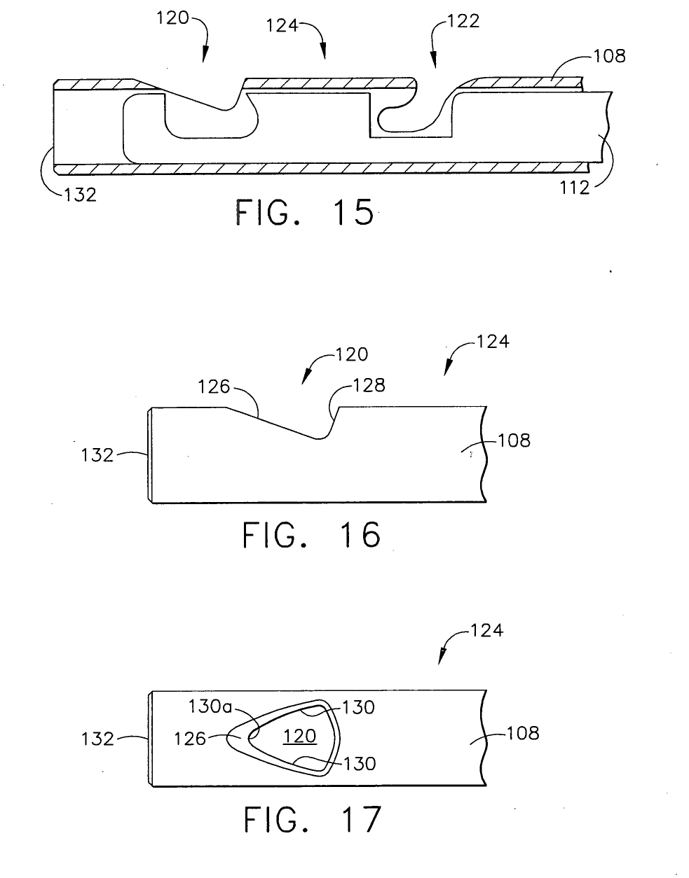

As cutter blade end 20 is advanced toward end 22 of tube 10, suture 40 is eventually urged against edge 30a by sharp edge 36. As sharp edge 36b advances past cutting edge 30a to a position so as to occlude slot 24 adjacent edge 30a, with suture 40 being cut as the shaft 14 is moved from the first position to the position shown in FIG. 10, with the end of severed segment 40a of suture 40 being captured as shown in FIG. 10. Since the radial clearance between shaft 14 and tube 10 is less than the diameter of suture 40, the end of severed segment 40a does not maintain its original circular cross sectional shape between shaft 14 and tube 10, but is deformed, such as becoming frayed.

Edge 32a and edge 36a together form a pair, with shaft 14 and tube 10 being configured to retain severed segment 40a adjacent to the pair in the position illustrated in FIG. 10. The pair of edge 30a and edge 36b are configured to sever segment 40a as shown.

As can be seen, grip 8 and knob 12 are configured such that end 20 of shaft 14 does extends beyond edge 30a. Preferably, end 20 does not extend beyond end 22 of tube 10, keeping sharp edge 36 protected.

As described, the axial offset edges 30a and 32a function in combination with cutter blade end 20, the clearance between distal end portion 38 and the internal diameter of tube 10 at distal end portion 26 and suture 40 to initiate capture of suture 40 prior to the cutting action beginning. In this depicted embodiment of the invention, the severed suture segment is captured by a portion extending generally in an axial direction within the interior of the tube, disposed between the shaft and the tube. Within the teaching of this invention, cutting may be initiated before end 20 reaches capture edge 32a, as long as capture of suture 40 is accomplished before severing suture 40.

The same may be accomplished with edges which are not aligned (i.e., not axially offset) as seen in FIG. 11, which is an alternate embodiment of end 20 and slot 24. In FIG. 11, the slot terminates in symmetrical axial portions having longitudinally aligned edges 42a and 42b. Diametrically opposite sides 44a and 44b of cutter blade end 44 of shaft 46 are longitudinally (axially) offset relative to each other, creating the offset between capture and cut as previously described. As shaft is advanced to cut suture 40, capture edge 44a contacts suture 40 first, pinching it between shaft 46 and the interior of tube 48 past edge 42a resulting in retention of the severed portion of suture 40. Cutting edge 44b is sharp as a result of the curved configuration of shaft 46 forming an angle at edge 44b of less than 90° at that location, and cuts suture 40 as it passes edge 42b.

As described thus far, suture cuter 2 includes an axial offset between the initial engagement of the suture on one side, between an end edge of the slot and the end of the cutter blade, and the initial engagement of the suture on another side, between an end edge of the slot and the end of the cutter blade.

In yet another embodiment, a suture cutter according to the teachings of the present invention may also be constructed with the offset between the cutting edge and the capture edge being reversed such that initiation of the cutting occurs prior to initiation of the capture. FIG. 12 illustrates such an alternate embodiment, which may advantageously be used with a monofilament suture 50. In this embodiment, capture edge 52a of axial portion 52 of slot 54 is located closer to end 56 than cutting edge 58b of axial portion 58 of slot 54. As shaft 60 is advanced to cut, edge 62b of end 62 contacts suture 50 and urges it against edge 58a prior to being urged against edge 52a and 62a. A monofilament suture cannot bend as much as a multifilament suture, and suture 50 is cut between edge 58a and end 62. Preferably prior to suture 50 being completely severed, the end being severed 50a is engaged by advancing edge 62a, urging and capturing it against capture edge 52a. The axial travel of shaft 60 is such that edge 62a of end 62 captures severed suture 50a, but is the travel is limited to stop short of cutting severed suture 50a.

Although in the embodiment illustrated, tube 10 and shaft 14 are rigid, they may be flexible as long as the distal end of suture cutter 2 has sufficient rigidity to function according to the teachings of the present invention. Although tube 10 and shaft 14 are circular, they may be any suitable complementary shape.

Although stainless steel suture material is specifically discussed herein, it will be understood that the present invention is not limited to use with stainless steel, and may be used with any suture material, such as polymer, gut, or absorbable material.

By way of non-limiting example, a suture cutter as described above for use in arthroscopic surgery was constructed of material suitable for tissue contact, with a tube outer diameter of 0.120 and a cutter shaft outside diameter of 0.093, having 0.002 clearance between it and the inside diameter of the tube at the distal end, for use with a 2-0 stainless steel braided suture having a nominal diameter of 0.013 inches, comprised of 49 strands.

Another embodiment of the present invention is depicted in FIGS. 13-24. FIG. 13 is a top view of combined suture bead & knot cutter and suture cutter assembly 100, and FIG. 14 is a cross sectional side view thereof taken along line 14-14 of FIG. 11. Assembly 100 includes tube assembly 102 and cutter assembly 104. Tube assembly 102 comprises grip 106 secured to one end of hollow tube 108 by any suitable means, such as by a set screw. Cutter assembly 104 includes knob 110 secured to one end of shaft 112 by any suitable means, such as by a set screw.

Grip 106 includes recess 114 configured to receive extension 116. In this embodiment, recess 114 is not cylindrical so that the orientation of extension 116 aligns the features located at distal end 118 of shaft 112 properly relative to opening 120 and slot 122 at distal end 124 of tube 108, as also seen in FIG. 13. Slot 122 comprises part of the suture cutter portion of the combined assembly 100, the construction and operation of which is discussed above, while opening 120 comprises part of the suture bead & knot cutter portion, which will be described in detail. It is noted that although the suture bead & knot cutter is disclosed herein as part of a single tool in combination with a suture cutter, the suture bead & knot cutter may comprise a single tool just as the suture cutter may comprise a single tool, as previously described.

Referring to FIGS. 15-17, opening 120 is formed through tube 108 between end 132 of tube 108 and slot 122. As will be appreciated, suture cutter slot 122 may alternatively be disposed between end 132 and opening 120. Opening 120 is formed through tube 108, depicted as having two inclined surfaces 126 and 128 disposed generally perpendicular to each other. In the embodiment depicted, surface 126 is disposed 20° relative to the axis of tube 108. This produces opening 120 with the shape shown generally in FIG. 17. Edge 130 comprises the edge formed between surface 126 with the interior of tube 108, having cutting edge 130a disposed at the apex of opening 120 closest to end 132.

End 132 is open in this embodiment. Opening 120 is configured to receive therethrough an enlargement formed on or carried by a suture, such as a bead or knot. A stop member is also referred to herein as a bead. As used herein, suture enlargement refers to any structure secured to or mounted on a suture, as well as any configuration of a suture, which is larger than the diameter of the suture.

Referring to FIGS. 18 and 19, there is shown a side view and top view of distal end 118 of shaft 112, which includes suture bead and knot cutter blade portion 134 and suture cutter blade portion 136. The relative spacing between suture bead and knot cutter blade portion 134 and suture cutter blade portion 136 locates both at the initial pre-cut position (FIG. 15) relative to opening 120 and slot 122, respectively. A hole (not shown) through grip 106 similar to hole 11 of grip 8, or a detent structure may be provided to assist the operator in properly locating suture bead and knot cutter blade portion 134 and suture cutter blade portion 136 to use either.

Slot 122 includes edges which are not axially offset with respect to each other, with the offset feature of the suture cutter being formed in suture cutter blade 136. As seen in FIG. 19, suture cutter blade 136 includes opposite sides 136a and 136b which are longitudinally (axially) offset relative to each other, creating the offset between capture and cut as described above. FIG. 21 illustrates an enlarged side view of suture cutter blade 136.

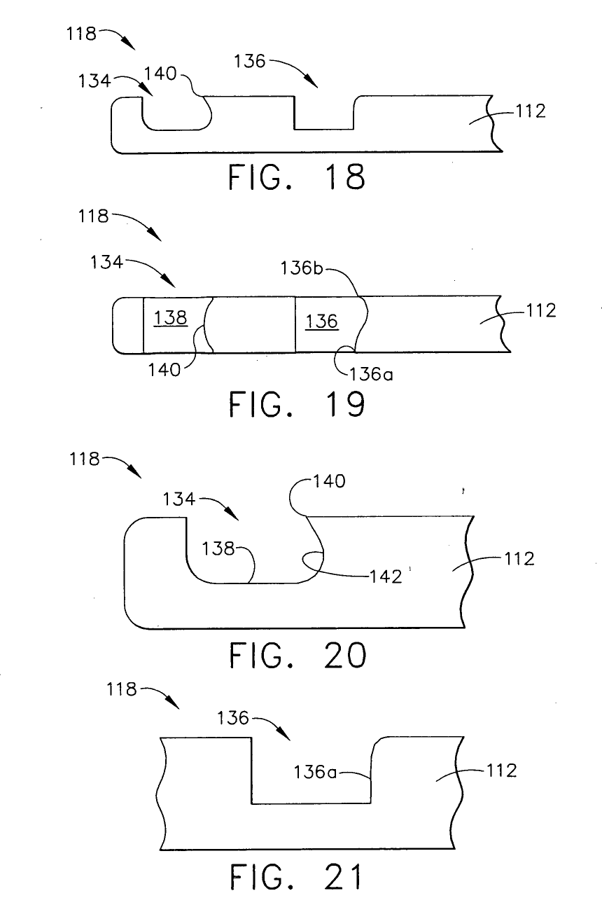

Referring to FIG. 20, suture bead and knot cutter blade portion 134 includes generally flat portion 138, and overhanging cutting edge 140 which forms recess 142. The depth of flat portion 138 aligns suture 148 with cutting edges 130a and 140.

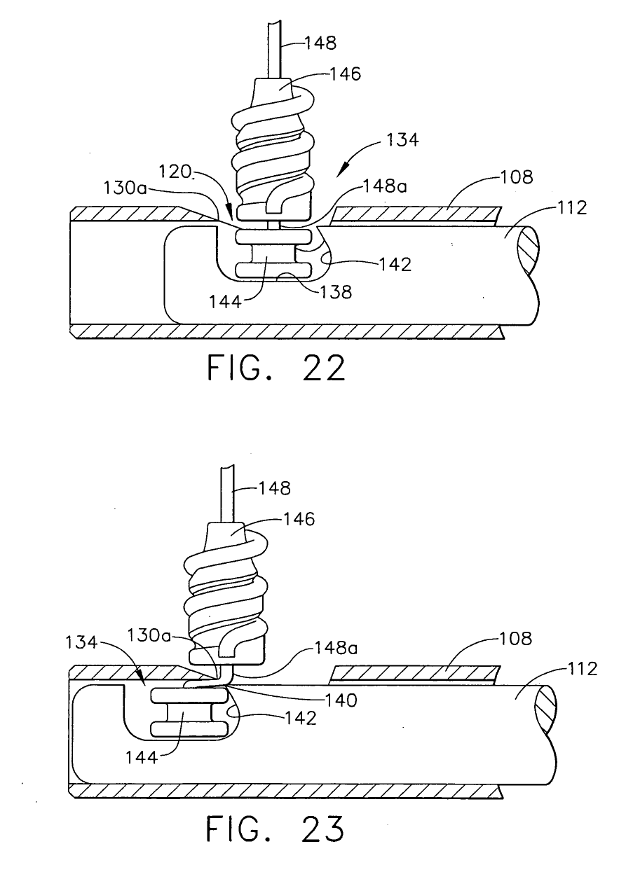

Referring to FIG. 22, suture bead cutting blade portion 134 is illustrated aligned with opening 120, with bead 144 disposed within the interior of tube 108, adjacent flat portion 138. When bead 144 is located toward cutting edge 130a, bead 144 is effectively captured inside of tube 108 due to the shape of opening 120 (see FIG. 15). Bead 144 may be axisymmetrical, although crimping bead 144 is known to produce a “C” cross section.

As shaft 112 is advanced to cut the portion 148a of suture 148 between bead 144 and anchor 146, suture 148 is urged toward and against cutting edge 130a, with bead 144 partially disposed in recess 142. Flat portion 138 helps to resist the rotation of bead 144, helping to keep suture 148 perpendicular to cutting edge 130a as the cut is initiated. FIG. 23 illustrates cutting edge 130a adjacent cutting edge 140 as severance of suture 148 at portion 148a is almost complete.

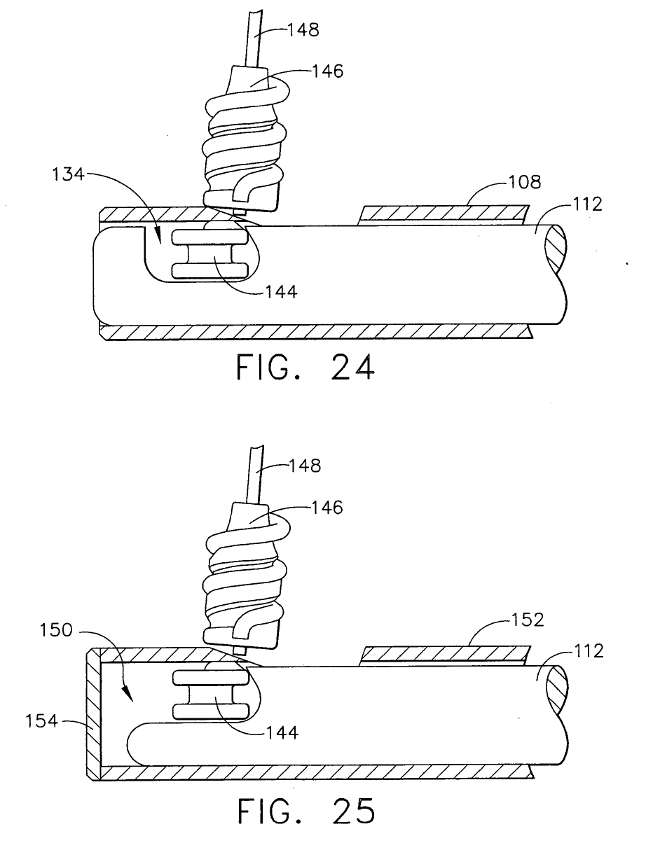

FIG. 24 illustrates bead 144 captured within suture bead cutting blade portion 134 after suture 148 has been cut. It will be appreciated by those of ordinary skill in the art that, instead of cutting the portion of suture 148 proximate to the suture enlargement, the suture enlargement itself may be cut.

FIG. 25 shows an alternate embodiment of suture bead cutting blade portion 150. In this embodiment, cutting blade portion 150 does not define an enclosed cavity within tube 152, but is open on the end. In order to retain bead 144 after suture 148 is cut, tube 152 includes end cap 154, welded to the end of tube 152.

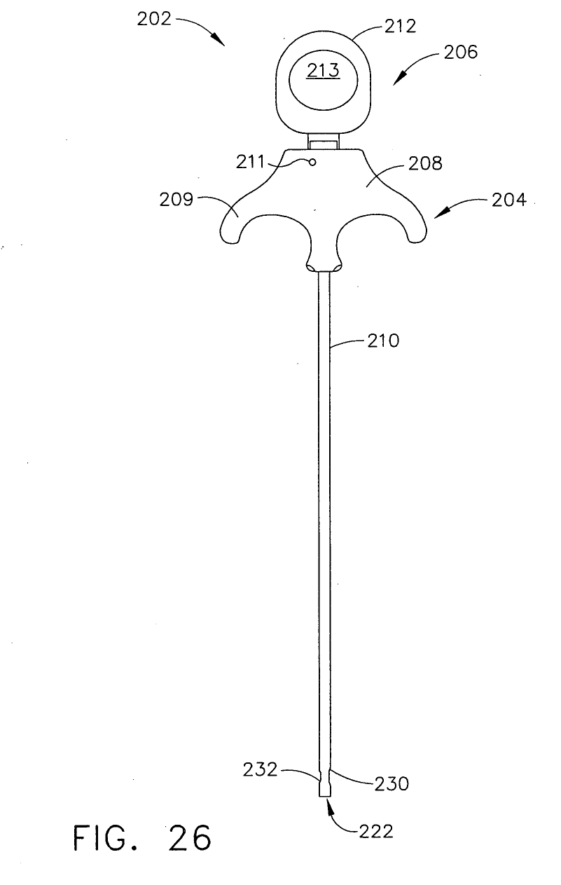

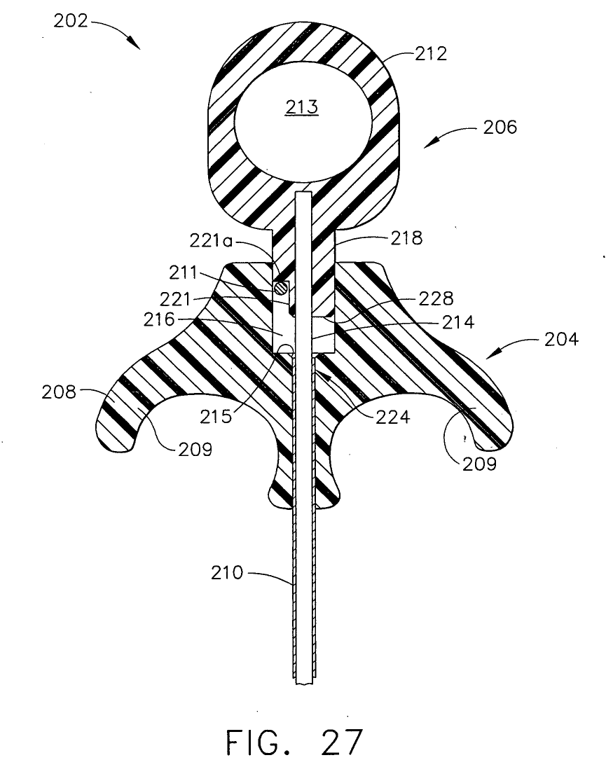

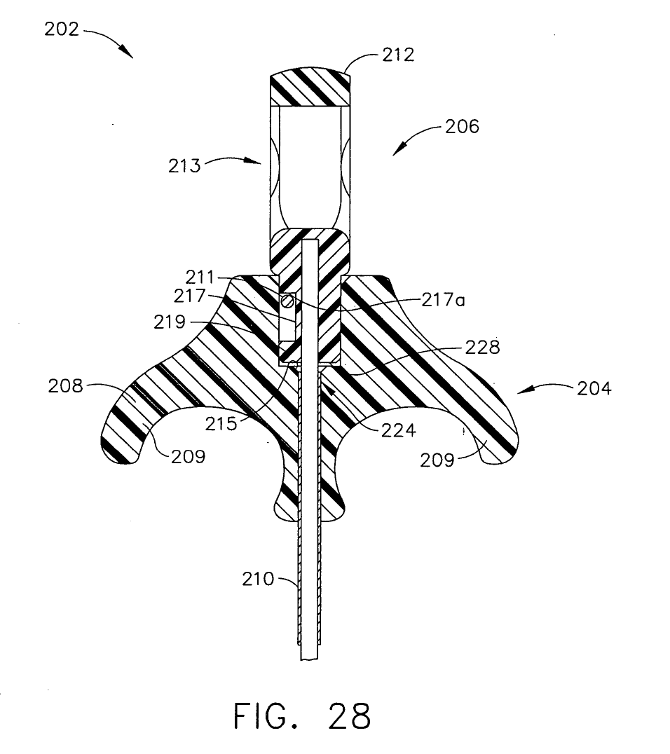

FIGS. 26 through 41 show yet more alternate embodiments of a suture cutting device. FIG. 26 is a top view of an alternate suture cutter assembly 202 constructed in accordance with teachings of the present invention. FIGS. 27 and 28 are cross sectional views taken along the axis of the device, parallel to the page of FIG. 26. Suture cutter assembly 202 includes tube assembly 204 and cutter assembly 206.

As shown in FIGS. 27 and 28, tube assembly 4 comprises grip 208 having two curved members 209 for the operator's fingers and cylindrical recess 216 with fixed pin 211 inside cylindrical recess 16. Pin 211 is oriented perpendicular to curved members 209. Grip 208 is secured to proximal end 224 of hollow tube 210, such that tube 210 may be accessed at the base of cylindrical recess 216. Edge 215 adjacent to tube 210 at base of cylindrical recess 216 is chamfered for guidance of shaft 214 into tube 210.

Cutter assembly 206 includes pusher 212 secured to proximal end of shaft 214. Pusher 212 includes opening 213 for the operator's thumb, for example, and extension 218. Extension 218 has first recessed flat 217 that is parallel to opening 213. First recessed flat 217 does not extend to base 228 of extension 218. Thus, first recessed flat 217 effectively provides flange 219 at base 228 of extension 218, though flange 219 does not extend beyond circumference of extension 218. Second recessed flat 221 is recessed perpendicular to first recessed flat 217. Unlike first recessed flat 217, second recessed flat 221 extends to base 228 of extension 218. Second recessed flat 221 has adjacent top surface 221 a that is axially lower on extension 218 than corresponding top surface 217a adjacent to first recessed flat 217.

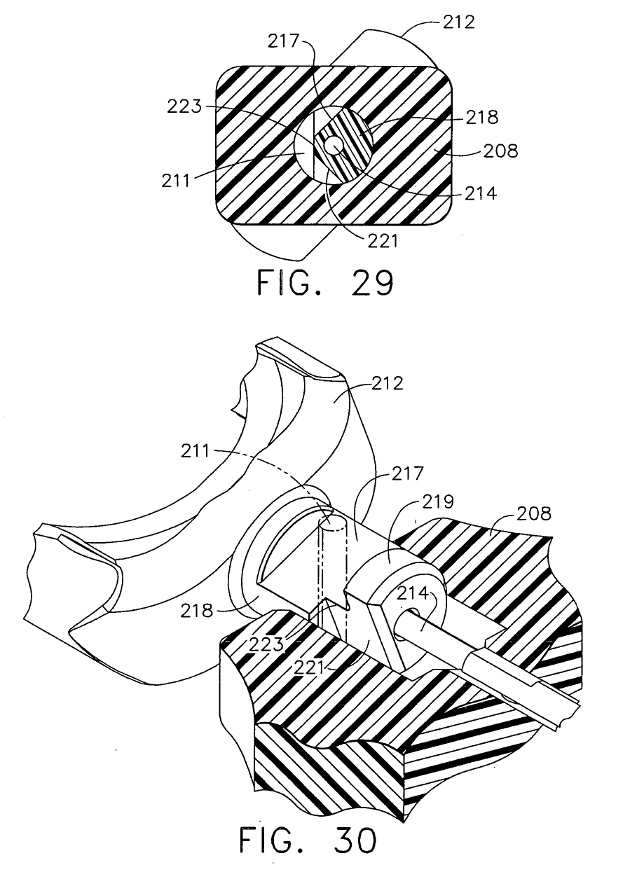

As shown in FIGS. 29 and 30, the intersection of first recessed flat 217 and second recessed flat 221 provides contact surface 223. Contact surface 223 may be rounded (e.g. has a radius). Alternatively, contact surface 223 may comprise a chamfered or beveled edge, such as the surface shown in FIG. 29, or a sharp corner, such as the corner shown in FIG. 30, at the intersection of first recessed flat 217 and second recessed flat 221.

Recess 216 in grip 208 is configured to receive extension 218 of pusher 212. In the present example, pin 211 and extension 218 are configured such that opening 213 in pusher 212 must be parallel to curved members 209 of grip 208 in order for any part of extension 218 to be disposed within recess 216 below pin 211. This requirement results from second recessed flat 221 providing necessary initial clearance of pin 211 for extension 218. However, it will be appreciated that tube assembly 204 or cutter assembly 206 may be configured such that any or no particular orientation of opening 213 is required for substantial disposal of extension 218 within recess 216.

In the present example, with extension 218 initially disposed within recess 216, further advancement of extension 218 into recess 216 is prevented by engagement of pin 211 with top surface 221a adjacent to second recessed flat 221. This configuration, shown in FIG. 27, where top surface 221a is so engaged with pin 211, will hereinafter be referred to as suture cutter assembly 202 being in “the pushing configuration,” and will be described in further detail below. In order to advance extension 218 further into recess 216 beyond the pushing configuration in the present example, pusher 212 must be rotated approximately 90° relative to grip 208, such that opening 213 is perpendicular to curved members 209. Contact surface 223 is configured to provide resistance to such rotation by providing interference with pin 211. This interference may be created by configuring extension 218 such that the minimum distance between contact surface 223 and the wall of recess 216 is 0.006 to 0.008 inches less than the diameter of pin 211. Of course, extension 218 or pin 211 may be configured to provide more or less interference, including no interference at all.

The configuration shown in FIG. 28, where pusher 212 has been rotated such that opening 213 is perpendicular to curved members 209 while extension 218 is disposed within recess 216 will hereinafter be referred to as suture cutter assembly 202 being in “cutting mode,” and will be described in further detail below. A rounded contact surface 223 may provide a relatively smooth transition from the pushing configuration to cutting mode. Transition stages between the pushing configuration and cutting mode are shown in FIGS. 29 and 30. It will be appreciated that, with suture cutter assembly 202 in cutting mode, extension 218 may be reciprocated within recess 216. It will be further appreciated that such reciprocation will be upwardly axially limited by engagement of pin 211 with flange 219. Thus, in the present example, pin 211 and flange 219 prevent pusher 212 from being fully withdrawn from grip 208 during cutting mode. However, pusher 212 or grip 208 may be configured to permit full withdrawal of pusher 212 from grip 208 during cutting mode. Such permission may be granted by, for example, eliminating flange 219 or pin 211.

In the present example, grip 208 and pusher 212 are each a molded plastic. By way of example, Delran or any other suitable plastic may be used. Alternatively, grip 208 or pusher 212 may be made of any other suitable material or by any suitable method. In the present example, tube 210 is secured to grip 208 by molding grip 208 with tube 210 predisposed in the mold. Similarly, shaft 214 is secured to pusher 212 by molding pusher 212 with shaft 214 predisposed in the mold. Any exterior part of tube 210 or shaft 214 that will be within molded grip 208 or pusher 212, respectively, may be knurled or grit-blasted to promote attachment. Alternatively, it will be appreciated that tube 210 may be secured to grip 208, and shaft 214 may be secured to knob 212, by respective set screws. Of course, any other suitable means for securing them together may be used.

In the embodiment depicted in FIG. 26, shaft 214 is rigid and solid, and dimensioned to reciprocate within rigid tube 210. The clearance between shaft 214 and tube 210 provides some resistance to axial movement of shaft 214. End 222 of tube 212 has a distal edge 222a. Preferably, distal edge 222a is rounded. However, it will be appreciated that other configurations of distal edge 222a may be used.

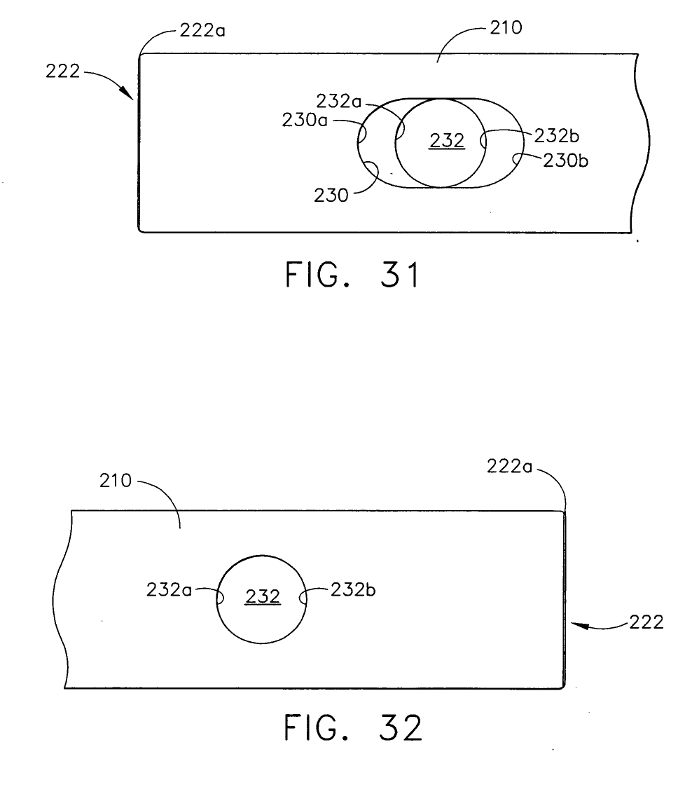

Referring also to FIGS. 31 and 32, apertures 230 and 232 are formed through the sidewall of tube 210 at distal end portion 226 of tube 210, which communicate with the interior defined by tube 210. Apertures 230 and 232 are shown as aligned and opening to the same direction in which curved members 29 extend, providing an index to the relative orientation between grip 208 and apertures 230 and 232. However, apertures 230 and 232 may open in any direction relative to grip 208.

As shown in FIG. 31, one aperture 230 is larger than the other aperture 232. While smaller aperture 232 is generally circular, larger aperture 230 is generally oblong circular, extending along axis of tube 210. In the present example, apertures 230 and 232 are centered on the same axis. Distal edge 230a of larger aperture 230 is disposed closer to end 222 of tube 210 than distal edge 232a of smaller aperture 232. Proximal edge 230b of larger aperture 30 is disposed further from end 22 of tube 10 than proximal edge 232b of smaller aperture 232. It will be appreciated that edges 230a or 230b of larger aperture 230 or edges 232a or 232b of smaller aperture 232 may be positioned differently along axis of tube 210 than what is shown in the figures. Apertures 230 and 232 may be formed by any well-known process, such as by milling or electro discharge machining, by way of example.

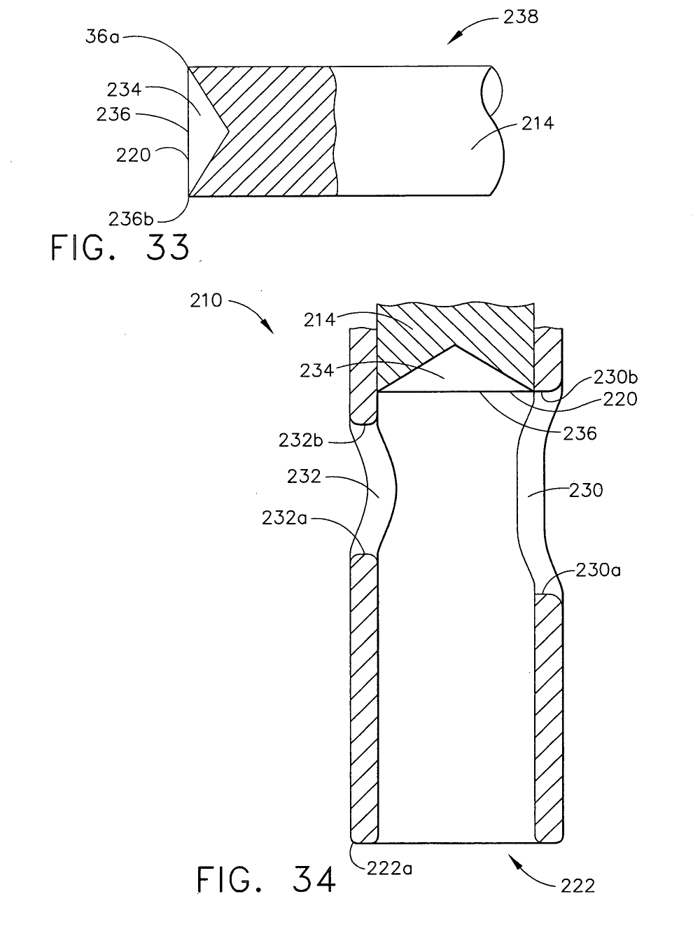

As seen in FIG. 33, end 220 of shaft 214 comprises the cutter blade, which in the embodiment depicted comprises conical shape depression 234 forming an annular sharp edge 236 about the periphery of end 220 of distal end portion 238. Edge 236 comprises edge 236a and edge 236b.

When suture cutter assembly 202 is in the pushing configuration, as shown in FIG. 34 for the present example, end 220 of shaft 214 will preferably be substantially aligned with proximal edge 230b of larger aperture 230. Thus, in the pushing configuration, end 220 preferably does not occlude any portion of apertures 230 and 232. Accordingly, suture 240 may be passed through both apertures 230 and 232 when suture cutter assembly 202 is in the pushing configuration. Of course, it will be appreciated that shaft 214 may be configured such that there is some distance between end 220 of shaft 214 and the nearest proximal edge 230b or 232b of aperture 230 or 232, respectively, when suture cutter assembly 202 is in the pushing configuration. Likewise, suture cutter assembly 202 may be configured such that, in the pushing configuration, shaft 214 occludes a portion of one or both apertures 230 and/or 232; but shaft 214 will preferably not completely occlude both apertures 230 and 232 when suture cutter assembly 202 is in the pushing configuration.

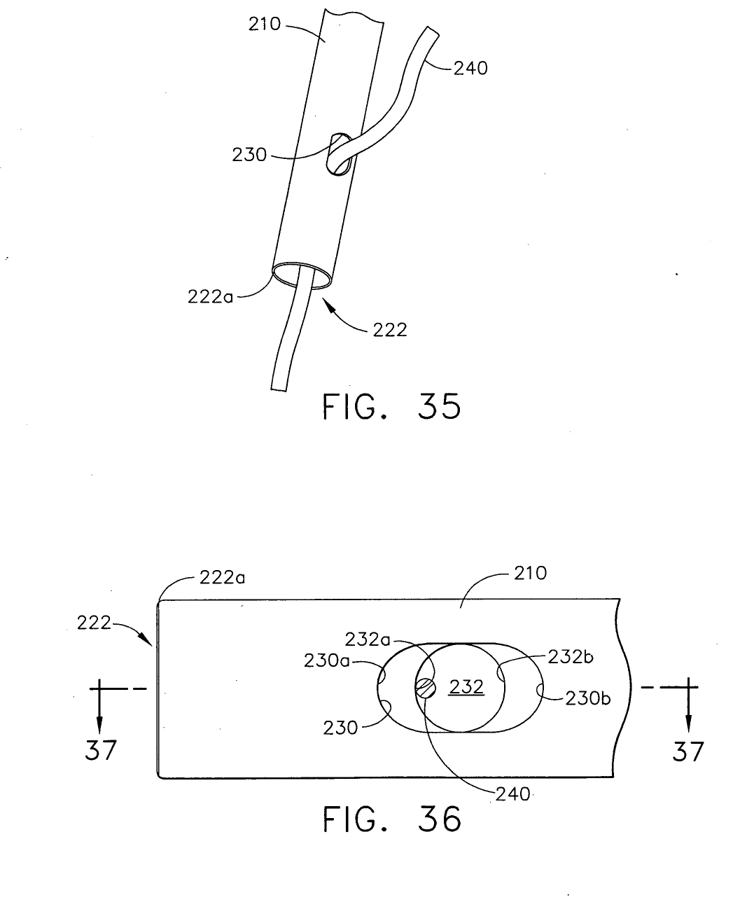

When suture cutter assembly 202 is in the pushing configuration, suture cutter assembly 202 may be used to push knots of sutures, as may be desired to tighten such knots. By way of example, where suture 240 has a loose knot, a free end of suture 240 may be passed through open end 222 of tube 210, then through aperture 230 as shown in FIG. 35. With suture cutter assembly 202 in the pushing configuration, end 220 of shaft 214 may act as a guide for the free end of suture 240 as it travels through tube 210 by blocking suture 240 from traveling further up tube 210, thereby guiding the free end of suture 240 to and through aperture 230. When a sufficient length of suture 240 has passed through aperture 230, the operator may grip the free end of suture 240 by any suitable means. Next, the operator may engage distal edge 222a of tube 210 with the knot, then slide end 222 of tube 210 along suture 240 so as to push the knot in suture 240. Those of ordinary skill in the art will appreciate that such pushing may serve to tighten the knot. It will be appreciated that, by making distal edge 221a of tube 210 rounded or otherwise smooth, unnecessary wear of suture 240 during such pushing may be avoided.

Referring to FIGS. 36-39, a process for cutting suture 240 with suture cutter assembly 202 is illustrated. Suture 240 is passed through apertures 230 and 232 so that suture 240 is disposed in apertures 230 and 232, with end 220 and sharp edge 236 initially located up from apertures 230 and 232, away from suture 240, so that end 220 does not occlude any portion of apertures 230 and 232 (e.g., when suture cutter assembly 202 is in the pushing configuration). It will also be appreciated that suture 240 may be passed through both apertures 230 and 232 when suture cutter assembly 202 is in cutting mode, so long as shaft 214 has not been axially advanced far enough through tube 210 as to be prohibitively occlusive of aperture 230 or 232. With suture 240 disposed in both apertures 230 and 232, and to the extent that suture cutter assembly 202 is not already in cutting mode, pusher 212 is then rotated 90° to place suture cutter assembly 202 in cutting mode.

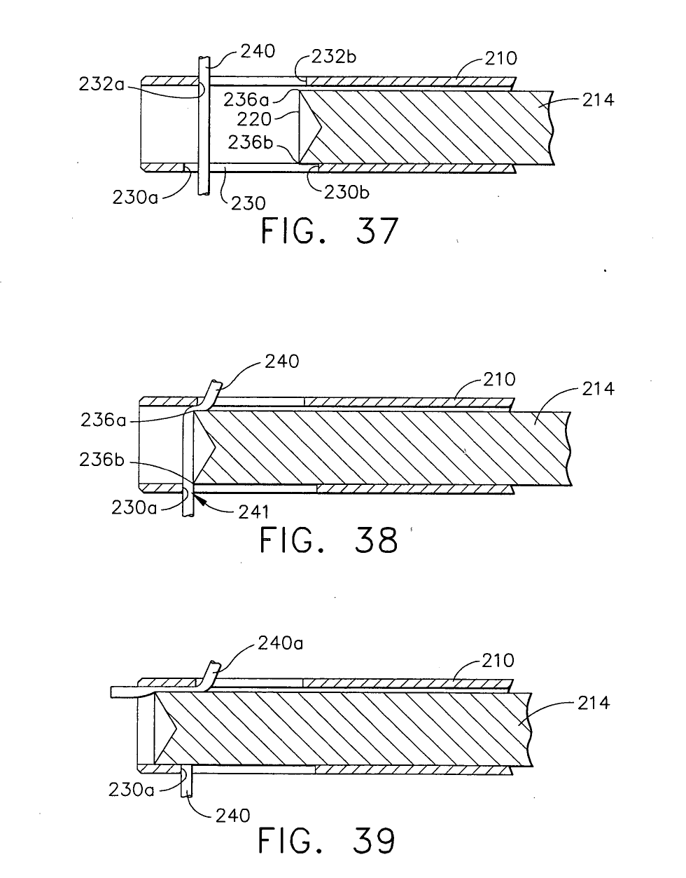

As seen in FIGS. 36 and 37, suture 240 is shown disposed adjacent edge 232a, generally perpendicular to the longitudinal axes of tube 210 and shaft 214. However, it will be appreciated that suture 240, while disposed in apertures 230 and 232 may be spaced from or adjacent to both edges 230a and 232a, until shaft 214 is urged into contacted with suture 240.

In FIG. 38, shaft 239 has been moved from the first position shown in FIG. 37, urged into contact with suture 240, with sharp edge 236a of end 220 disposed adjacent edge 232a, and occluding aperture 232 adjacent edge 232a. The transverse clearance, also referred to as diametrical clearance, between shaft 214 adjacent edge 236a and the sidewall of tube 210 adjacent edge 232a, has a dimension which is less than a corresponding dimension of the suture 240, such that a portion of suture 240 is captured or retained between the interior surface the sidewall of tube 210 and shaft 214 but larger than a dimension what would sever suture 240. This configuration makes edge 232a the capture edge of aperture 232. A bend is formed in suture 240 by this action, and shaft 214 and sharp edge 236b are concomitantly urged against the opposite side of the interior of tube 210, the portion adjacent cutting edge 230a.

At the position shown in FIG. 38, gap 241, extending longitudinally, is defined between edge 230a and edge 236b. At the position shown in FIG. 38, gap 241 has a longitudinal dimension that is smaller than the corresponding dimension of suture 240. Gap 241 may be smaller than the corresponding dimension when aperture 232 adjacent edge 232a is initially occluded by end 220.

As cutter blade end 220 is advanced toward end 222 of tube 210, suture 240 is eventually urged against edge 230a by sharp edge 236. As sharp edge 236b advances past cutting edge 230a to a position so as to occlude aperture 230 adjacent edge 230a, with suture 240 being cut as the shaft 214 is moved from the first position to the position shown in FIG. 39, with the end of severed segment 240a of suture 240 being captured as shown in FIG. 39. Since the radial clearance between shaft 214 and tube 210 is less than the diameter of suture 240, the end of severed segment 240a does not maintain its original circular cross sectional shape between shaft 214 and tube 210, but is deformed, such as becoming frayed.

Edge 232a and edge 236a together form a pair, with shaft 214 and tube 210 being configured to retain severed segment 240a adjacent to the pair in the position illustrated in FIG. 39. The pair of edge 230a and edge 236b are configured to sever segment 240a as shown.

As can be seen in FIG. 39, grip 208 and pusher 212 are configured such that end 220 of shaft 214 extends beyond edge 230a. Preferably, end 220 does not extend beyond end 222 of tube 210, keeping sharp edge 236 protected.

As described, the axial offset edges 230a and 232a function in combination with cutter blade end 220, the clearance between distal end portion 238 and the internal diameter of tube 210 at distal end portion 226 and suture 240 to initiate capture of suture 240 prior to the cutting action beginning. In this depicted embodiment of the invention, the severed suture segment is captured by a portion extending generally in an axial direction within the interior of the tube, disposed between the shaft and the tube. Within the teaching of this invention, cutting may be initiated before end 220 reaches capture edge 232a, as long as capture of suture 240 is accomplished before severing suture 240.

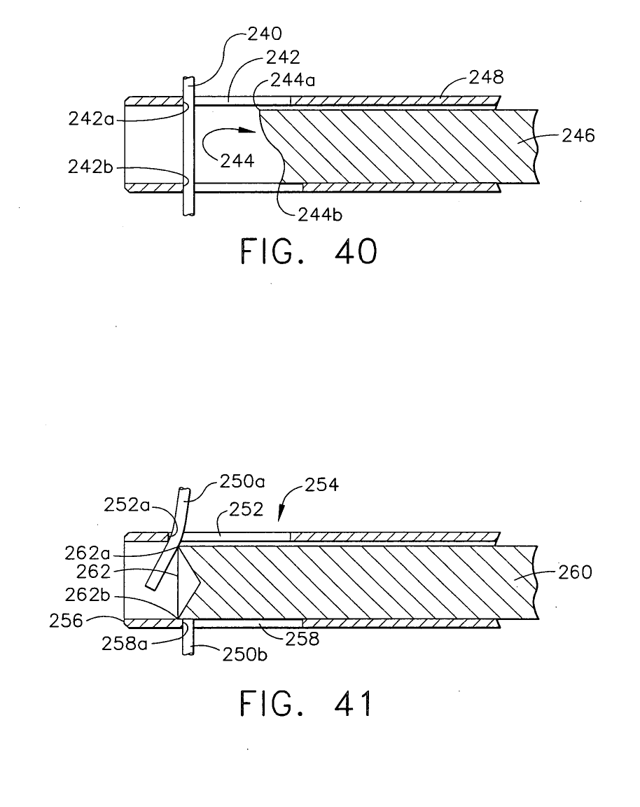

The same may be accomplished with distal aperture edges that are aligned (i.e., not axially offset) as seen in FIG. 240, which is an alternate embodiment of end 244 of shaft 246 and apertures 242. In FIG. 40, apertures 242 have longitudinally aligned distal edges 242a and 242b. Diametrically opposite sides 244a and 244b of cutter blade end 244 of shaft 246 are longitudinally (axially) offset relative to each other, creating the offset between capture and cut as previously described. As shaft is advanced to cut suture 240, capture edge 244a contacts suture 240 first, pinching it between shaft 246 and the interior of tube 248 past edge 242a resulting in retention of the severed portion of suture 240. Cutting edge 244b is sharp as a result of the curved configuration of shaft 246 forming an angle at edge 244b of less than 90° at that location, and cuts suture 240 as it passes edge 242b.

As described thus far, suture cutter 202 includes an axial offset between the initial engagement of the suture on one side, between an end edge of one aperture and the end of the cutter blade, and the initial engagement of the suture on another side, between an end edge of the other aperture and the end of the cutter blade.

Thus, it will be appreciated that apertures 230 and 232 need not be sized or shaped differently. It will also be appreciated that apertures 230 and 232 need not be aligned on a common axis. By way of example, one aperture may be located closer to distal end 222 of tube 210 than another equally-sized aperture located on the other side of the axis of tube 210.

In yet another embodiment, a suture cutter according to the teachings of the present invention may also be constructed with the offset between the cutting edge and the capture edge being such that initiation of the cutting occurs prior to initiation of the capture. FIG. 41 illustrates such an alternate embodiment, which may advantageously be used with a monofilament suture 250. In this embodiment, capture edge 252a of aperture 252 is located closer to end 256 than cutting edge 258a of aperture 258. As shaft 260 is advanced to cut, edge 262b of end 262 contacts suture 250 and urges it against edge 258a prior to being urged against edge 252a and 262a. A monofilament suture cannot bend as much as a multifilament suture, and suture 250 is cut between edge 258a and end 262b. Preferably, prior to suture 250 being completely severed, the end being severed 250a is engaged by advancing edge 262a, urging and capturing it against capture edge 252a. The axial travel of shaft 260 is such that edge 262a of end 262 captures severed suture 250a, but is the travel is limited to stop short of cutting severed suture 250a.

Although in the embodiment illustrated, tube 210 and shaft 214 are rigid, they may be flexible as long as the distal end of suture cutter 202 has sufficient rigidity to function according to the teachings of the present invention. Although tube 210 and shaft 214 are circular, they may be any suitable complementary shape.

Although stainless steel suture material is specifically discussed herein, it will be understood that the present invention is not limited to use with stainless steel, and may be used with any suture material, such as, by way of example only, polymer, gut, or absorbable material.

By way of non-limiting example, a suture cutter as described above for use in arthroscopic surgery was constructed of material suitable for tissue contact, with a tube outer diameter of 0.120 and a cutter shaft outside diameter of 0.093, having 0.002 clearance between it and the inside diameter of the tube at the distal end, for use with a 2-0 stainless steel braided suture having a nominal diameter of 0.013 inches, comprised of 49 strands. The smaller aperture has a generally uniform diameter of 0.070. The larger aperture has a smallest dimension of 0.070, with the distance between edges comprising the elongated portion being 0.115. The distal edge of the larger aperture is 0.022 distally further than the distal edge of the smaller aperture. Of course, other suitable dimensions may be used.

As an alternative, non-limiting example, a suture cutter as described above may be constructed with a tube outer diameter of 0.120 and a cutter shaft outside diameter of 0.070, having 0.002 clearance between it and the inside diameter of the tube at the distal end. Of course, other suitable dimensions may be used.

In summary, numerous benefits have been described which result from employing the concepts of the invention. The foregoing description of a preferred embodiment of the invention has been presented for purposes of illustration and description. It is not intended to be exhaustive or to limit the invention to the precise form disclosed. Obvious modifications or variations are possible in light of the above teachings. The embodiment was chosen and described in order to best illustrate the principles of the invention and its practical application to thereby enable one of ordinary skill in the art to best utilize the invention in various embodiments and with various modifications as are suited to the particular use contemplated. It is intended that the scope of the invention be defined by the claims appended hereto.

1. A suture cutter for severing a first portion of a suture from a second portion of the suture, said suture cutter comprising:
a. a first member defining an interior, said first member comprising:
i. a side wall; and
ii. one or more openings formed through said side wall, said one or more openings being configured to receive the suture, said one or more openings comprising first and second spaced apart edges; and
b. a second member disposed at least partially within said interior and moveable relative to said first member from a first position to a second position, said second member comprising:
i. a third edge which forms a first pair with said first edge, said first and second members being configured to retain the first portion adjacent said first pair when said second member is in said second position; and
ii. a fourth edge which forms a second pair with said second edge, said fourth edge and said second edge being configured to sever said first portion from said second portion as said second member is moved from said first position to said second position.

2. The suture cutter of claim 1, wherein said first and second edges are disposed relative to each other and said third and fourth edges are disposed relative to each other such that, as said second member moves from said first position to said second position, the suture is urged against said second and fourth edges prior to the suture being urged against said first and third edges.

3. The suture cutter of claim 2, wherein said first pair is configured to retain the first portion when said second member is disposed in said second position.

4. The suture cutter of claim 2, wherein said first pair is configured to exert sufficient force on the first portion to retain the first portion prior to the suture being completely severed.

5. The suture cutter of claim 1, wherein said first and second edges are disposed relative to each other and said third and fourth edges are disposed relative to each other such that, as said second member moves from said first position to said second position, the suture is urged against said first and third edges prior to the suture being urged against said second and fourth edges.

6. The suture cutter of claim 5, wherein said first and second members are configured to retain the first portion by retaining at least part of the first portion within said interior between said side wall and said second member.

7. The suture cutter of claim 5, wherein said first and second members are dimensioned to provide clearance between said side wall adjacent said first edge and said second member adjacent said third edge sufficient to dispose at least part of the first portion within said clearance thereby retaining the first portion.

8. The suture cutter of claim 7, wherein said first and second members are dimensioned to urge said fourth edge against said side wall adjacent said second edge when at least part of the first portion is disposed in said clearance.

9. The suture cutter of claim 1, wherein said one or more openings comprises a slot with a J configuration.

10. The suture cutter of claim 1, wherein said third and fourth edges comprise one end of said second member.

11. The suture cutter of claim 1, wherein said second member comprises a first end, said third and fourth edges being spaced from said first end.

12. The suture cutter of claim 1, wherein said first and second pairs are disposed relative to each other such that said first portion is retained by said first pair as said second member is moved toward said second position prior to said first portion being completely severed from said second portion by said second pair.

13. The suture cutter of claim 1, wherein said first member further comprises an open distal end having a first distal edge, said open distal end being configured to permit passage of an end of said suture through said open distal end and one of said one or more openings, said first distal edge being configured to engage with a knot in said suture for pushing said knot.

14. The suture cutter of claim 13, wherein said first distal edge is chamfered.

15. The suture cutter of claim 13, wherein said first distal edge is rounded.

16. The suture cutter of claim 13, wherein said first member further comprises a grip, wherein said second member further comprises a pusher, wherein said grip and said pusher are configured to be selectively oriented in a plurality of positions, said plurality of positions comprising a pushing configuration and a cutting mode.

17. The suture cutter of claim 16, wherein said second member further comprises a closed distal end, wherein said closed distal end is configured to guide said end of said suture through said one of said one or more openings when said grip and said pusher are in said pushing configuration.

18. The suture cutter of claim 1, wherein said one or more openings includes an opening configured to receive at least a first portion of a suture enlargement, wherein said second member further comprises a suture enlargement cutting portion, said suture enlargement cutting portion comprising a recess formed in said second member and a blade portion adjacent said recess, said recess being configured to receive at least a second portion of said suture enlargement, said blade portion being configured to cut at least one of said suture enlargement or a portion of said suture proximate to said suture enlargement.

19. The suture cutter of claim 18, wherein said suture enlargement cutting portion defines an enclosed cavity within said first member.

20. The suture cutter of claim 18, wherein said first member has a closed distal end.

21. The suture cutter of claim 1, said one or more openings comprising a pair of apertures.

22. A suture cutter for severing a first portion of a suture from a second portion of the suture, said suture cutter comprising:
a. a first member comprising:
i. a side wall which defines an interior, said interior extending in a first direction; and
ii. one or more openings configured to receive the suture, said one or more openings comprising first and second edges spaced apart transversely relative to said first direction;
b. a second member disposed at least partially within said interior and moveable relative to said first member along said first direction from a first position to a second position, said second member comprising third and fourth edges spaced apart transversely relative to said first direction, said third edge forming a first pair with said first edge and said fourth edge forming a second pair with said second edge; and
c. the opening of said one or more openings adjacent said first edge being occluded when said second member is disposed at said second position, said second pair defining a gap extending in said first direction between said second and fourth edges when said second member is disposed at said second position.

23. The suture cutter of claim 22, wherein said gap has a dimension when said second member is disposed at said second position which is smaller than a corresponding dimension of the suture.

24. The suture cutter of claim 22, wherein said opening adjacent said first edge is initially occluded by movement of said second member toward said second position, and wherein said gap having a dimension which is smaller than a corresponding dimension of the suture when said opening adjacent said first edge is initially occluded.

25. The suture cutter of claim 22, wherein said first and second edges are offset relative to each other in said first direction.

26. The suture cutter of claim 22, wherein said third and fourth edges are offset relative to each other in said first direction.

27. The suture cutter of claim 22, wherein said one or more openings comprises a slot.

28. The suture cutter of claim 22, wherein said one or more openings comprises a pair of apertures.

29. The suture cutter of claim 22, wherein said one or more openings comprises first and second transversely spaced portions extending generally in said first direction, said first spaced portion terminating in said first edge, said second spaced portion terminating in said second edge.

30. The suture cutter of claim 22, including transverse clearance between said side wall adjacent said first edge and said second member adjacent said third edge, said clearance being less than a corresponding dimension of the suture and larger than a dimension that would sever the suture.

31. The suture cutter of claim 22, wherein said second member is moveable from said first position to a third position, said second position being intermediate said first and third positions, an opening of said one or more openings adjacent said second edge being occluded when said second member is disposed at said third position.

32. The suture cutter of claim 22, wherein said first member further comprises an open distal end having a first distal edge, said open distal end being configured to permit passage of an end of said suture through said open distal end and one of said one or more openings, said first distal edge being configured to engage with a knot in said suture for pushing said knot.

33. The suture cutter of claim 32, wherein said first distal edge is chamfered.

34. The suture cutter of claim 32, wherein said first distal edge is rounded.

35. The suture cutter of claim 32, wherein said first member further comprises a grip, wherein said second member further comprises a pusher, wherein said grip and said pusher are configured to be selectively oriented in a plurality of positions, said plurality of positions comprising a pushing configuration and a cutting mode.

36. The suture cutter of claim 35, wherein said second member further comprises a closed distal end, wherein said closed distal end is configured to guide said end of said suture through said one of said one or more openings when said grip and said pusher are in said pushing configuration.

37. The suture cutter of claim 22, wherein said one or more openings includes an opening configured to receive at least a first portion of a suture enlargement, wherein said second member further comprises a suture enlargement cutting portion, said suture enlargement cutting portion comprising a recess formed in said second member and a blade portion adjacent said recess, said recess being configured to receive at least a second portion of said suture enlargement, said blade portion being configured to cut at least one of said suture enlargement or a portion of said suture proximate to said suture enlargement.

38. The suture cutter of claim 37, wherein said suture enlargement cutting portion defines an enclosed cavity within said first member.

39. The suture cutter of claim 37, wherein said first member has a closed distal end.

40. The suture cutter of claim 22, said one or more openings comprising a pair of apertures.

41. A suture cutter for severing a first portion of a suture from a second portion of the suture, said suture cutter comprising:
a. a first pair comprising a first edge and first surface and a second edge and second surface, said first edge being moveable in a first direction relative to said second edge from a first position at which there exists a first gap extending in said first direction between said first and second edges to a second position, said first pair configured to initially engage the suture between said first edge and said second edge as said first edge is moved from said first position to said second position, said first pair configured to retain the first portion adjacent said first pair when said first edge is in said second position;
b. a second pair comprising a third edge and a fourth edge, said third edge being moveable in said first direction relative to said fourth edge from a third position at which there exists a second gap extending in said first direction between said third and fourth edges to a fourth position at which said second gap does not exist, concomitantly with movement of said first edge from said first position to said second position, said second pair configured to initially engage the suture between said third and fourth edges as said third edge is moved from said first position to said second position, said second pair configured to sever the first portion from the second portion as said third edge is moved from said third position to said fourth position;
c. an offset in said first direction between initial engagement of the suture by said first pair and initial engagement of the suture by said second cooperating pair.

42. The suture cutter of claim 41, wherein said offset comprises said first edge being offset in the first direction from said third edge.

43. The suture cutter of claim 41, wherein said offset comprises said third edge being offset in the first direction from said fourth edge.

44. The suture cutter of claim 41, wherein said second pair initially engages the suture as said third edge is moved from said third position to said fourth position prior to said first pair initially engaging the suture as said first edge is moved from said first position to said second position.

45. The suture cutter of claim 44, wherein said first gap has a dimension when said first edge is disposed at said second position which is smaller than a corresponding dimension of the suture so that said first and second edges are urged against the first portion thereby retaining the first portion.

46. The suture cutter of claim 41, wherein said first pair initially engages the suture as said first edge is moved from said first position to said second position prior to said second pair initially engaging the suture as said third edge is moved from said third position to said fourth position.

47. The suture cutter of claim 46, wherein said first gap does not exist when said first edge is disposed at said second position.

48. The suture cutter of claim 47, comprising clearance in a direction transverse to said first direction between said first surface and said second surface when said first edge is disposed at said second position, said clearance being configured to retain the at least part of the first portion.

49. A suture cutter for severing a first portion of a suture from a second portion of the suture, said suture cutter comprising:
a. a tube comprising:
i. a side wall defining an interior extending longitudinally along an axis, said side wall having an interior surface; and
ii. one or more openings configured to receive the suture, said one or more openings extending through said side wall in communication with said interior, said one or more openings comprising first and second spaced apart distal edges;
b. a shaft disposed at least partially in said interior and moveable along said axis in a first direction relative to said tube from a first position to a second position, said shaft comprising:
i. a first end;
ii. an outer surface; and
iii. third and fourth transversely spaced apart edges disposed adjacent said outer surface;
c. said first and third edges forming a first cooperating pair configured to engage the suture disposed in said one or more openings as said shaft is moved from said first position to second position;
d. said second and fourth edges forming a second cooperating pair configured to engage the suture disposed in said one or more openings as said shaft is moved from said first position to said second position; and
e. an axial offset between initial engagement of the suture by said first cooperating pair and initial engagement of the suture by said second cooperating pair.

50. The suture cutter of claim 49, wherein said axial offset comprises said first edge being axially offset from second edge.

51. The suture cutter of claim 50, wherein said first end comprises third and fourth edges.

52. The suture cutter of claim 49, wherein said axial offset comprises said third edge being axially offset from said fourth edge.

53. The suture cutter of claim 52, wherein said first end comprises third and fourth edges.

54. The suture cutter of claim 52, wherein said third and fourth edges are spaced from said first end.

55. The suture cutter of claim 49, wherein said first cooperating pair is configured to sever the first portion from said second portion as said shaft is moved to said second position.

56. The suture cutter of claim 55, wherein at least one of said one or more openings adjacent said first pair is occluded when said shaft is disposed at said second position.

57. The suture cutter of claim 56, comprising a gap between said second and fourth edges when said shaft is disposed at said second position, said gap configured to retain the first portion when said shaft is disposed at said second position.

58. The suture cutter of claim 56, wherein said second cooperating pair is configured to initially engage the suture prior to the first portion being severed.

59. The suture cutter of claim 49, wherein at least one of said one or more openings adjacent said first edge is occluded by said shaft when said shaft is disposed at said second position.

60. The suture cutter of claim 59, wherein said tube, said shaft and said first pair are configured to retain at least part of the first portion when said shaft is disposed at said second position.

61. The suture cutter of claim 60, comprising clearance between inner and outer surfaces adjacent said first pair, said clearance configured to retain said at least part of the first portion when said shaft is disposed at said second position.

62. The suture cutter of claim 61, wherein said shaft is urged against said inner surface adjacent said second pair by the at least part of the first portion retained by said clearance.

63. The suture of claim 59, wherein at least one of said one or more openings adjacent said second edge is occluded by said shaft when said shaft is disposed at said second position.

64. The suture cutter of claim 59, wherein said second cooperating pair is configured to sever the first portion from said second portion as said shaft is moved to said second position.

65. The suture cutter of claim 64, wherein, as said shaft is moved from said first position to said second position, at least one of said one or more openings adjacent said first edge is occluded by said shaft prior to the suture being initially contacted by said second pair.

66. The suture cutter of claim 49, wherein said tube further comprises an open distal end having a first distal edge, said open distal end being configured to permit passage of an end of said suture through said open distal end and one of said one or more openings, said first distal edge being configured to engage with a knot in said suture for pushing said knot.

67. The suture cutter of claim 66, wherein said first distal edge is chamfered.

68. The suture cutter of claim 66, wherein said first distal edge is rounded.

69. The suture cutter of claim 66, wherein said first member further comprises a grip, wherein said second member further comprises a pusher, wherein said grip and said pusher are configured to be selectively oriented in a plurality of positions, said plurality of positions comprising a pushing configuration and a cutting mode.

70. The suture cutter of claim 69, wherein said shaft further comprises a closed distal end, wherein said closed distal end is configured to guide said end of said suture through said one of said one or more openings when said grip and said pusher are in said pushing configuration.

71. The suture cutter of claim 49, wherein said one or more openings includes an opening configured to receive at least a first portion of a suture enlargement, wherein said shaft further comprises a suture enlargement cutting portion, said suture enlargement cutting portion comprising a recess formed in said shaft and a blade portion adjacent said recess, said recess being configured to receive at least a second portion of said suture enlargement, said blade portion being configured to cut at least one of said suture enlargement or a potion of said suture proximate to said suture enlargement.

72. The suture cutter of claim 71, wherein said suture enlargement cutting portion defines an enclosed cavity within said tube.

73. The suture cutter of claim 71, wherein said tube has a closed distal end.

74. The suture cutter of claim 49, said one or more openings comprising a pair of apertures.

75. A suture cutter for severing a first portion of a suture from a second portion of the suture, said suture cutter comprising:
a. a first member;
b. a second member, said second member being moveable in a first direction relative to said first member from a first position to a second position;
c. a first pair comprising a first edge carried by said first member and a first edge portion carried by said second member, wherein said first member, said second member and said first pair are configured such that said first pair retains the first portion of the suture disposed proximal said first pair when said second member is in said second position; and
d. a second pair comprising a second edge carried by said first member and a second edge portion carried by said second member, wherein said first member, said second member and said second pair are configured such that said second pair severs said first portion from the second portion of the suture when disposed proximal said second pair as said second member is moved to said second position.
